(19) United States
(12) Patent Application Publication
(10) Pub. No.: US 2022/0242753 A1
(43) Pub. Date: Aug. 4, 2022

(54) Immersible Water Purifier for Use with Flexible Water Bladders

(71) Applicant: David Lee, Hamilton (CA)

(72) Inventor: David Lee, Hamilton (CA)

(21) Appl. No.: 17/163,698

(22) Filed: Feb. 1, 2021

(51) Int. Cl. C02F 1/32; H05K 7/20; A61L 2/26; A61L 2/10

(52) U.S. Cl. CPC C02F 1/325; H05K 7/20509; H05K 1/18; A61L 2/10; A61L 2/26

(57) ABSTRACT

A UV water purifier for use with a water storage container is disclosed. The UV water purifier consists of a UV LED lamp mounted to a first portion of a heatsink having first and second portions, the UV LED lamp and the first portion of the heatsink being contained in a UV transparent portion of a housing. The housing is mounted to the water storage container by a bracket, the housing and bracket being configured to position the UV transparent portion of the housing and second portion of the heatsink within the water storage container such that the UV transparent portion of the housing is immersed in water and the second portion of the heat sink physically contacts the water when the water storage container is filled with water.

### FIELD OF THE INVENTION

The invention relates generally to ultraviolet water purifiers.

### BACKGROUND OF THE INVENTION

Water purifiers which use ultraviolet light to purify water have been widely available for many years. These purifiers generally consist of an ultraviolet lamp which is configured to shine ultraviolet light through a transparent wall into a volume of water. in some cases the water is carried through UV transparent tubes positioned adjacent to the UV lamp. In other designs the UV lamp is contained in a UV transparent enclosure which is surrounded by water. Regardless of which design used, the effectiveness of the UV purification is dependent on the intensity of the UV light being used and the duration the water is exposed to the UV light. Longer duration times and higher UV light intensity increases the purification effect of the UV light.

A key factor determining the intensity of the UV light generated by a UV lamp is the power output of the UV lamp. Unfortunately, highly efficient LED UV lamps are often limited in their power output by their susceptibility to overheating. The higher the voltage and current applied to the LED lamp, the higher the power, the more intensely the LIED lamp generates UV and the higher the temperature experienced by the LED lamp. When the temperature of the LED lamp exceeds a predefined limit, the LED lamp's performance starts to degrade and will eventually fail. Dissipating the heat generated by the LED lamp is often difficult given the confined spaces the LED lamp is usually placed in to ensure good UV exposure to the water. An improved design which maximizes the heat dissipation of the UV LED lamp while maximizing the amount of water being exposed to the UV light is therefore required.

### SUMMARY OF THE INVENTION

In accordance with one aspect of the present invention, there is provided a UV water purifier for use with a water storage container configured to store a volume of water. The UV water purifier consists of a UV LED lamp mounted to a first portion of a heatsink having first and second portions, the UV LED lamp and the first portion of the heatsink being contained in a UV transparent portion of a housing. The housing is mounted to the water storage container by a bracket, the housing and bracket being configured to position the UV transparent portion of the housing and second portion of the heatsink within the water storage container such that the UV transparent portion of the housing is immersed in water and the second portion of the heat sink physically contacts the water when the water storage container is filled with water.

In accordance with another aspect of the present invention, there is provided a UV water purifier for use with a water storage container having an opening, the water storage container configured to store a volume of water. The UV water purifier includes a first housing having a closed first end and a second end opposite the first end, the first housing being UV transparent. A second housing is provided having opposite first and second ends, the first end of the second housing being coupled to the second end of the first housing by a coupling mounting the first and second housings together. The coupling is made of a material having a high thermal conductivity. A UV LED lamp is disposed in the first housing, the UV LED lamp being thermally coupled to a heat sink made of a material having a high thermal conductivity. The heat sink has opposite first and second portions, the first portion configured to mount the UV LED lamp thereon and the second portion configured to form the coupling. A control unit is contained within the second housing for driving the UV LED lamp. Finally, a lid is provided which is configured to close off the opening of the water storage container, the lid being configured to mount the second housing within the opening of the water storage container, the lid, first and second housings and the coupling being configured such that the first housing and the thermally conductive coupling are both immersed in water when the storage container is filled with water.

With the foregoing in view, and other advantages as will become apparent to those skilled in the art to which this invention relates as this specification proceeds, the invention is herein described by reference to the accompanying drawings forming a part hereof, which includes a description of the preferred typical embodiment of the principles of the present invention.

### DESCRIPTION OF THE DRAWINGS

FIG. 1 is a perspective view of a water purifier made in accordance with the present invention mounted to a flexible water storage container.

FIG. 2 is a perspective view of the water purifier shown in FIG. 1 with the water purifier being detached from the flexible water storage container.

FIG. 3 is an exploded view of the water purifier shown in FIG. 1.

FIG. 4 is a sectional view of the water purifier shown in FIG. 1 showing the water purifier mounted in the water storage unit.

FIG. 5 is a side view of the water purifier of FIG. 1 partially submersed in water.

FIG. 6 is a sectional view taken along line A-A of FIG. 5.

FIG. 7 is an expanded view of part B of FIG. 6 above.

In the drawings like characters of reference indicate corresponding parts in the different figures.

### DETAILED DESCRIPTION OF THE INVENTION

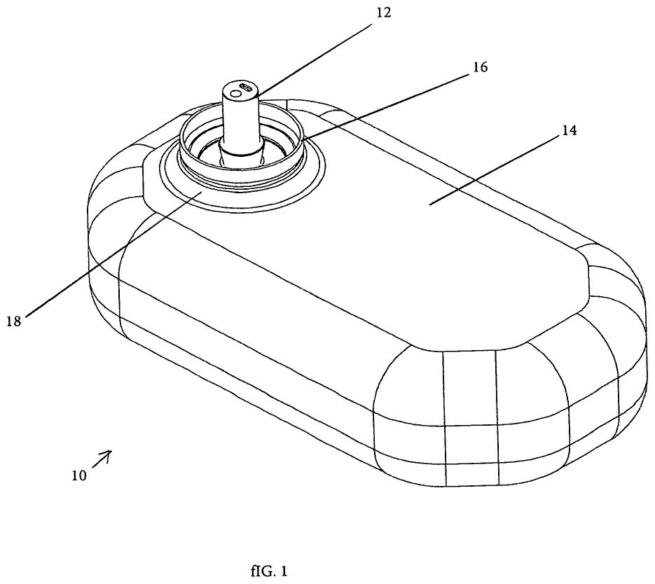

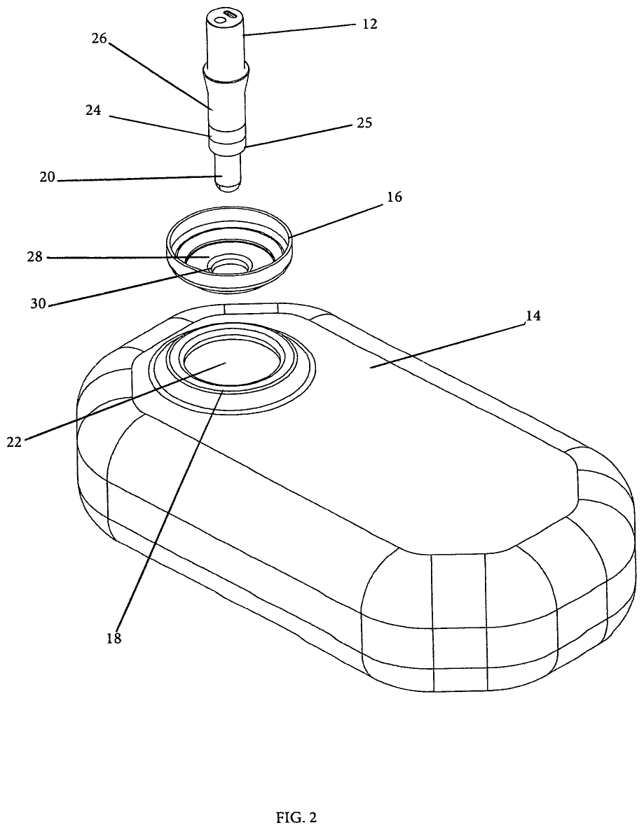

Referring to FIG. 1, a water purifier shown generally as item 10 includes a UV LED lamp unit 12 mounted to flexible water storage container 14 via mounting member 16. Mounting member 16 acts as a lid or cap to close off opening 18 in water storage container 14. Water storage container 14 consists of a container dimensioned to store a volume of water. Preferably water storage container 14 forms a flexible bladder like structure made of natural or artificial rubber, but it could be formed as a rigid container made of plastic or metal. Referring to FIG. 2, mounting element 16 positions UV LED lamp 12 such that lower portion 20 of the UV LED lamp projects into interior 22 of water storage container 14. UV LED lamp unit 12 has a midsection 26 which is dimensioned to fit tightly within aperture 30 formed on floor 28 of mounting member 16 such that the UV LED lamp unit is tightly held to the mounting member when attached. A pair of thermal conductor bands 24 and 25 are positioned below midsection 26 so that when UV LED lamp unit 12 is attached to mounting member 16 and the mounting member is screwed onto opening 18 to close off the opening, thermal conductor bands 24 and 25 are positioned within interior 22. Mounting member 16 and midsection 26 are dimensioned such that when water storage container 14 is filled with water, thermal conductor bands 24 and 25 will be immersed in the water and make physical contact with the water.

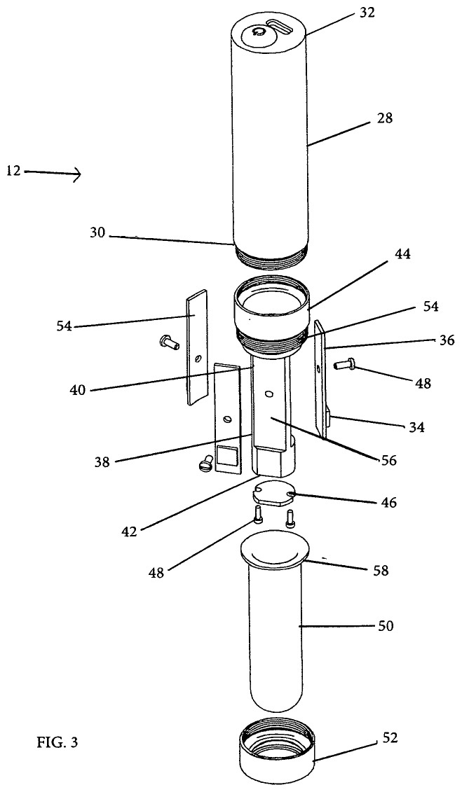

Referring now to FIG. 3, UV LED lamp unit 12 comprises a housing 28 having a threaded end 30 and a control end 32. Housing 28 encloses a drive circuit and battery for driving the UV LEDs making up the lamp. The recharging port and the on/off button are positioned at control end 32. Each of the UV LEDs 34 are mounted to elongated circuit boards 36, which are in turn mounted to elongated first portion 38 and heatsink 40. Heatsink 40 has opposite ends 42 and 44. End 44 is threaded to mate with threaded end 30 and forms a coupling joining heat sink 40 to housing 28. End 44 forms thermal conductor band 24 as see FIG. 2. Heatsink 40 has end 42 which has an additional circuit board 46 mounted thereto. Circuit boards 34 and 46 are mounted to heatsink 40 by fasteners 48. Circuit boards 34 have elongated flat surfaces 54 which are dimensioned to closely conform to flat surfaces 56 on heatsink 40 such that the length and width of the flat surfaces 54 and 56 are substantially equal. Flat surfaces 56 and 54 are very flat and smooth such that when they are mounted to each other there is good thermal contact between the two surfaces. A thermal paste may be applied to surfaces 54 and 56 to ensure excellent thermal contact between the circuit boards and the heatsink. Circuit boards 34 and 46 are made of a material having a high thermal conductivity. Heatsink 40 is formed as a single piece of material made of a material also having a high thermal conductivity. Transparent housing 50 is dimensioned to fit over first portion 38 of heatsink 40. Transparent housing 50 is preferably made of quartz or some other UV transparent material. Sealing ring 52 is provided to secure housing 50 to end 44 of heat sink 40. Sealing ring 52 has threads configured to mate with the threads of end 44. Transparent housing 50 has a flanged end 58 and sealing ring 52 is dimensioned and shaped to seal the flanged end to end 44 of heatsink 40 such that first portion 38 of heatsink 40 is contained within the transparent housing and the housing is rendered watertight.

Sealing ring 52 is also preferably made of a material having a high thermal conductivity such that when the sealing rind is threaded to end 54 of heatsink 40, there is good thermal contact between the sealing ring and the heatsink. Sealing ring forms thermal conductor band 25 as seen in FIG. 3. Since UV LEDs 34 are surface mounted to circuit boards 36 and 46 and since both the circuit boards and heatsink 40 are made of highly thermally conductive materials, heat generated by the UV LEDs is efficiently transmitted to thermal conductor bands 24 and 25. Thermal conductor bands 24 and 25 act as radiators to dissipate heat quickly, particularly if the thermal conductor bands are immersed in water. The material forming heatsink 40, circuit boards 36 and 46 and sealing ring 52 preferably has a thermal conductivity exceeding that of graphite. Suitable materials which can be used for heatsink 40, circuit boards 36 and 46 and sealing ring 52 include aluminum, copper, aluminum nitride, silicon carbide, silver, gold, graphite, alloys of aluminum, alloys of copper, alloys of silver and alloys of gold.

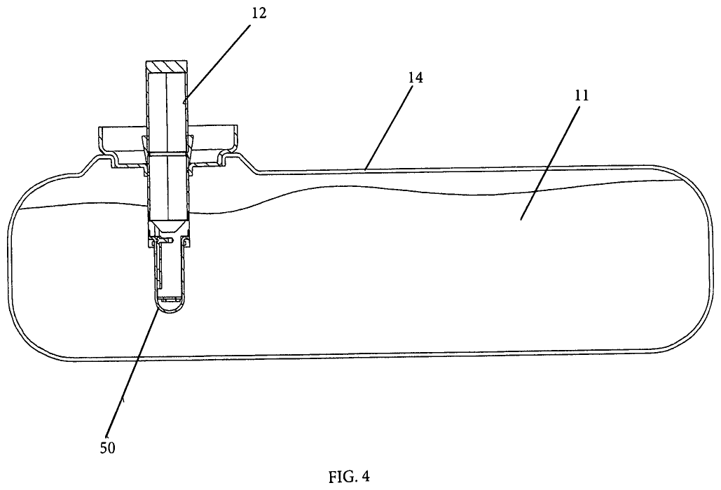

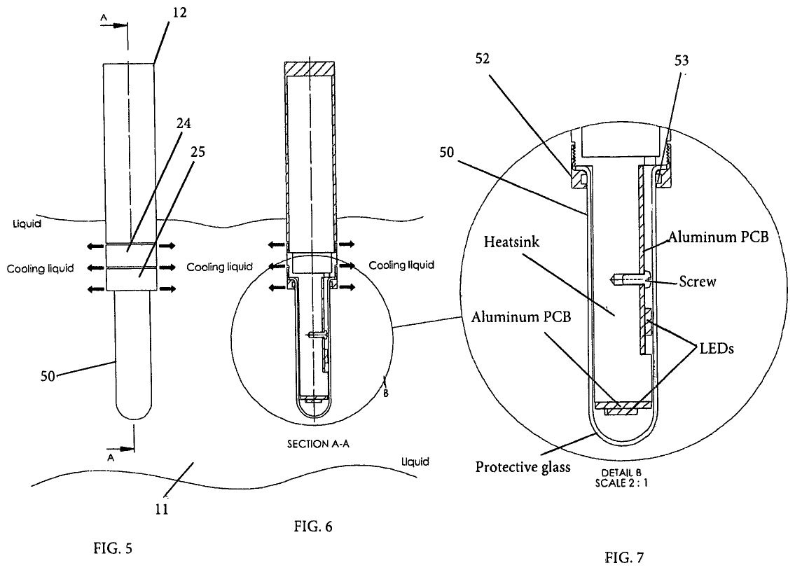

Referring now to FIG. 4, when mounted to container 14, transparent housing 50 of UV LED lamp unit 12 is positioned to be immersed in water 11 when the container is filled with water. When UV LED lamp unit 12 is activated, UV light is transmitted and passes through housing 50 and into the water to inactivate bacteria, microbes and viruses contained in the water. As best seen in FIGS. 5 and 6, thermal conductor bands 24 and 25 are immersed in water 11 such that the thermal conductor bands make direct physical contact with the water. Since thermal conductor bands 24 and 25 make direct physical contact with the water, and since the conductor bands are made of a material having a high thermal conductivity, heat generated from the operation of UV LED lamp 12 is efficiently dissipated into the water. This keeps the UV LEDs in the UV LED lamp cool, permitting the UV LEDs to be operated at a higher intensity than would be possible if there was not a direct thermal link between the LEDs and thermal conductor bands 24 and 25. This efficient heat dissipation permits UV LED lamp to purify the water more efficiently (i.e. render bacterial, microbes and viruses in the water inactive).

A specific embodiment of the present invention has been disclosed; however, several variations of the disclosed embodiment could be envisioned as within the scope of this invention. It is to be understood that the present invention is not limited to the embodiments described above, but encompasses any and all embodiments within the scope of the following claims

1. A UV water purifier for use with a water storage container having an opening, the water storage container configured to store a volume of water, the UV water purifier comprising:
a. A first housing having a closed first end and a second end opposite the first end, the first housing being UV transparent;
b. A second housing having opposite first and second ends, the first end of the second housing being coupled to the second end of the first housing by a coupling mounting the first and second housings together, the coupling being made of a material having a high thermal conductivity;
c. A UV LED lamp disposed in the first housing, the UV LED lamp being thermally coupled to a heat sink made of a material having a high thermal conductivity, the heat sink having opposite first and second portions, the first portion configured to mount the UV LED lamp thereon and the second portion configured to form the coupling;
d. A control unit contained within the second housing for driving the UV LED lamp, and
e. A lid configured to close off the opening of the water storage container, the lid being configured to mount the second housing within the opening of the water storage container, the lid, first and second housings and the coupling being configured such that the first housing and the thermally conductive coupling are both immersed in water when the storage container is filled with water.

2. The UV water purifier defined in claim 1 wherein the second end of the first housing is opened and further comprising a sealing ring for sealing the coupling unit to the fist end of the housing such that the first housing is made water tight, the sealing ring and coupling unit being complimentarily threaded, the sealing ring being made of a material having a high thermal conductivity such that the sealing ring and coupling unit are thermally coupled to each other when the sealing ring is threaded onto the coupling unit, the coupling unit, first housing and lid being further configured such that the sealing ring is immersed in water when the storage container is filled with water.

3. The UV water purifier defined in claim 2 wherein the heat sink comprises a continuous elongated member with the first portion of the heat sink having an elongated flat surface, the UV LED lamp comprising a UV LED mounted to an elongated PCB having a flat surface, the flat surface of the elongated PCB being thermally coupled to the flat surface of the heat sink, the elongated PCB being made of a metal having a high thermal conductivity.

4. The UV water purifier defined in claim 3 wherein the flat surface of the heatsink and the flat surface of the elongated PCB have matching widths and lengths, the flat surface of the elongated PCB being thermally sealed to the flat surface of the heat sink by thermal paste.

5. The UV water purifier defined in claim 4 wherein the first portion of the heatsink has three flat surfaces such that the first portion of the heat sink has a triangular cross-sectional profile and there are three separate UV LED lamps thermally mounted onto the heatsink.

6. The UV water purifier defined in claim 5 wherein the first portion of the heatsink has a flat terminal end, a fourth UV LED lamp being mounted on the flat terminal end, the fourth UV LED lamp comprising a UV LED mounted to a flat PCB made of a metal having a high thermal conductivity.

7. The UV water purifier defined in claim 1 wherein the material forming the heatsink and the coupling is selected from the group of materials comprising aluminum, copper, aluminum nitride, silicon carbide, silver, gold, graphite, alloys of aluminum, alloys of copper, alloys of silver and alloys of gold.

8. The UV water purifier defined in claim 2 wherein the first housing comprises an elongated cylindrical tube having an outside diameter, the first end of the first housing having a rim with a diameter slightly greater than the outside diameter of the elongated cylindrical tube, the sealing ring comprising an annular member with a shoulder portion having an inside diameter slightly greater than the outside diameter of the elongated tube but slightly less than the diameter of the rim, a sealing gasket being interposed in the shoulder.

9. A UV water purifier for use with a water storage container configured to store a volume of water, the UV water purifier comprising a UV LED lamp mounted to a first portion of a heatsink having first and second portions, the UV LED lamp and the first portion of the heatsink being contained in a UV transparent portion of a housing, the housing being mounted to the water storage container by a bracket, the housing and bracket being configured to position the UV transparent portion of the housing and second portion of the heatsink within the water storage container such that the UV transparent portion of the housing is immersed in water and the second portion of the heat sink physically contacts the water when the water storage container is filled with water.

10. The UV water purifier defined in claim 9 wherein the housing comprises the UV transparent portion coupled to a control housing by a coupling, the coupling being formed at least in part by the second part of the heatsink, the control housing dimensioned to contain a drive configured to drive the UV LED lamp.

11. The UV water purifier defined in claim 10 wherein the UV LED lamp comprises a UV LED mounted to a PCB having a flat surface dimensioned and configured to mount to a corresponding flat surface on the first portion of the heatsink, the PCB being made of a material having a high thermal conductivity.

12. The UV water purifier defined in claim 11 wherein the first portion of the heatsink has a plurality of flat sides, a separate UV LED lamp being mounted to each of the flat sides of the first portion of the heatsink.

13. The UV water purifier defined in claim 12 further comprising a sealing member for mounting the heat sink within the UV transparent portion of the housing such that the UV transparent portion of the housing remains water tight, the sealing member and the second portion of the heat sink being configured to thread together, the sealing member being made of a material having a high thermal conductivity and the sealing member is thermally coupled to the heat sink, the sealing member being dimensioned and configured to be in contact with the water when the water storage container is filled with water.

14. The UV water purifier defined in claim 13 wherein the second portion of the heat sink has a radiative surface where the heat sink contacts the water when the water storage container is filled with water, the sealing member having a radiative surface where the sealing member contacts the water, the radiative surface of the heat sink and the radiative surface of the sealing member combining to increase the total surface for heat dispersion into the water from the UV LED lamps.
